(19) United States
(12) Patent Application Publication
(10) Pub. No.: US 2024/0075285 A1
(43) Pub. Date: Mar. 7, 2024

(54) DEVICES, SYSTEMS AND METHODS USING FLEXIBLE CIRCUITRY FOR STIMULATING A BODY OF A SUBJECT TO CAUSE A DESIRED MOVEMENT

(71) Applicant: Andrew Matthew Dalhousie BRODIE, Kingsford NSW (AU)

(72) Inventor: Andrew Matthew Dalhousie BRODIE, Kingsford NSW (AU)

(21) Appl. No.: 18/271,744

(22) PCT Filed: Jan. 11, 2022
(86) PCT No.: PCT/AU2022/050007

§ 371 Date: Jul. 11, 2023

(30) Foreign Application Priority Data
Jan. 13, 2021 (AU) 2021900064

(51) Int. Cl. A61N 1/36; A61B 5/00; A61B 5/11; A61N 1/04; H05K 1/02; H05K 1/03

(52) U.S. Cl. CPC A61N 1/36014; A61B 5/0051; A61B 5/1114; A61B 5/112; A61B 5/6802; A61B 5/6831; A61N 1/0452; A61N 1/0456; A61N 1/0484; A61N 1/36003; H05K 1/0283; H05K 1/0393

(57) ABSTRACT

A stimulation device for guiding movement of a part of a body of a subject is provided. The device comprises a flexible substrate configured to at least partially conform to an outer surface of the part of the body, in use. The device further comprises a stretchable stimulator circuit that is at least partially embedded within the flexible substrate. The stimulator circuit comprises at least one stimulator configured to provide a stimulus to a part of a body to cause movement. A system for guiding movement of a subject is also provided including the stimulation device, a controller operably connected to the stimulation device for controlling the stimulation device, at least one sensor for sensing monitoring data, and a processor for comparing the obtained monitoring data with pre-determined movement information to determine whether a desired movement has occurred.

### FIELD OF THE INVENTION

The present invention relates to devices, systems and methods for providing stimulus and in particular to devices, systems and methods for providing stimulus to guide movement of a subject.

The invention has been developed primarily for use in guiding movement to achieve a desired movement and/or reduce undesirable movement by externally providing stimulus to a body. However, it will be appreciated that the invention is not limited to this particular field of use.

### BACKGROUND OF THE INVENTION

Neural synchrony is the simultaneous or synchronous oscillations of membrane potentials in a network of neurons connected with electrical synapses. Neural synchronisation is thought to play an important role in perceptual processes including being a conceptual correlate for consciousness. Neural synchrony has been widely suggested as an important way to understand how highly dimensional neural populations may be organised into lower level functional networks. Neural synchrony further provides flexibility in movement. The functional networks may be dynamically reconfigured so that collectives can form and then be quickly dissolved and rearranged as required.

In healthy people and during well learned movements (for example the sit-to-stand transition) neural synchrony between individual skeletal muscle activations across and cortical regions of interest may be measured using electrophysiology and analysed using signal processing algorithms. Higher neural synchrony may indicate the shared neural control of functional networks comprising multiple skeletal muscles. The result being the coordinated movement of multiple limbs to achieve a target outcome with little apparent conscious involvement.

In people living with neuromuscular diseases, motor impairment or movement disorders, or during the acquisition of new skills, the Central Nervous System (CNS) and Peripheral Nervous System (PNS) control may have to be reconfigured and new functional networks established and optimised. Until the target movements are rehabilitated or mastered, suboptimal neural control may result in less coordinated multi-limb movements that may require greater conscious involvement to complete.

Patients suffering from Parkinson's disease typically exhibit impaired gait. Such patients typically experience limb tremor and, in turn, freezing-of-gait (FOG).

Providing external cues to the body to guide movement of a patient has been successful in improving gait in patients with Parkinson's disease. Visual, auditory and mechanical cues have been used in various forms to provide therapy.

Tactile cues have been used in several research papers to provide therapy to patients with Parkinson's disease. Vibrators have been used to provide rhythmic tactile cues to the soles of feet to initiate walking with normal gait.

However, limitations of auditory cues include that they may not be suitable across a range of patients with Parkinsons disease.

Limitations of vibrating motors include that they are generally large and bulky, not precisely positioned.

Application of continuous vibration (e.g. for several seconds) to a muscle or body area such as feet, has also been investigated. However, it has shown limited effect in reducing freezing-of-gait and improve walking ability.

Vibrating platforms have also been used as a training device to build muscle mass and may reduce postural sway when someone stands on them. However, continuous vibration may alter proprioception, or increase the excitability of the muscle fibres, but it has not been shown to specifically guide the timing or amplitude of target movements.

The application of a single vibrator providing rhythmic, intermittent vibration or one-off vibration pulses has been tested. In an example, a single vibrating motor was attached to the wrist of a subject, and vibrations are repeated using a cadence usually within +−10% of normal cadence. This method was shown to be of limited value relative to providing auditory cues.

In “The attentional cost of external rhythmical cues and their impact on gait in Parkinson's disease: effect of cue modality and task complexity” (2007) J Neural Transm (Vienna), 114, 1243-8. by Rochester et al., the mechanism of a single vibrating motor was used as a substitute for an external auditory cue. Location of the vibration was not considered to be important, and vibrators were applied at the wrist of a user. The method included establishing a cadence for the user to follow. However, limitations included that the user was required to concentrate on following the external cue. and it is not an automated process.

U.S. patent application Ser. No. 15/638,115 discloses a wearable device comprising a plurality of actuators. The actuators in the wearable device are adjustable relative to one another in terms of their position and in various examples, the actuators may be adjusted in terms of duty cycle, power and/or position based on sensor data.

U.S. Pat. No. 9,943,250 discloses a method and system for provoking gait disorders, such as freezing of gait. The invention discloses display of situations calculated to cause freezing of gait which are presented to a subject and identifying incipit freezing of gait using changes in gait parameters. The invention discloses a portable device which detects incipit freezing of gait only.

Other known preventative measures for freezing-of-gait including the use of walking sticks, lasers projections on the floor, walkers and other devices that can be used to guide movement. However, these measures necessarily require concentration and manual operation by the user and are not automated processes.

Patent publications WO2017185050A1, US20160367191A1, WO2017120063A1 and U.S. Pat. No. 9,186,092B2 discloses various sensor systems and methods for monitoring and evaluating gait. However, these inventions are primary focused on monitoring various parameters associated with gait and, collecting and processing data.

The application of electrical stimulus has been shown to effectively reduce FOG. For example, in “Sensory Electrical Stimulation Cueing May Reduce Freezing of Gait Episodes in Parkinson's Disease” (2018) Journal of Healthcare Engineering, Article ID 4684925, by Rosenthal et al., the mechanism of “a fixed rhythmic sensory electrical stimulation (sES) cueing strategy” was used to reduce the time taken to complete walking tasks and a number of FOG episodes. However, the electrical stimulation was only applied at a fixed 700 ms duty cycle to a single body part.

In “Using smart socks and rhythmic haptic cues to stimulate the foot arch may reduce gait variability during a freezing of gait elicitation task” (2018) 1st International Motor Impairment Conference, 26-28 November, (P10), p66 by Brodie et al. and “Vibrating socks to improve gait in Parkinson's disease” (2019) Parkinsonism and Related Disorders 69 (2019) 59-60 by Koopman et al, it has also been shown that the application of haptic or vibrational stimulus applied to both feet may improve mobility or reduce FOG. In each document, a vibrotactile buzzer or vibrating coin motor attached to each foot arch and secured with a sock was tested.

The present invention seeks to provide a solution which will overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE INVENTION

In an aspect of the present disclosure, there is provided a stimulation device for guiding movement of a part of a body of a subject, the device comprising:
a flexible substrate configured to at least partially conform to an outer surface of the part of the body, in use;
a stimulator circuit at least partially embedded within the flexible substrate, the stimulator circuit comprising at least one stimulator configured to extracorporeally provide stimulation to the part of the body to guide movement.

Regarding the each stimulating device, the flexible substrate may comprise an orthosis.

The flexible substrate may comprise a wearable garment, wearable apparel or wearable accessory.

The flexible substrate may be configured to provide a uniform contact pressure, in use.

The flexible substrate may be stretchable in one or more directions. For example, the flexible substrate may be configured to stretch longitudinally and/or laterally. In an embodiment, the flexible substrate may comprise a stretchable bandage.

The substrate may be made of neoprene or a similar material. For example, the substrate may comprise neoprene rubber or stretchable velcro.

The stimulator circuit may be at least partially flexible.

The stimulator circuit may be stretchable.

The stimulator circuit may be configured to at least partially stretch in one or more directions. For example, the stimulator circuit may be configured to at least partially stretch longitudinally and/or laterally.

The stimulator circuit may comprise stretchable electrical conductors extending between electrical components.

The stretchable electrical conductors may be moveable between a reduced configuration and an extended configuration.

In an embodiment, the stretchable electrical conductors may comprise conductive wires in a reduced or non-linear arrangement within an elastic substrate.

The stretchable electrical conductors may comprise conductive wires in a reduced or non-linear arrangement so as to extend in a direction in which the elastic substrate is stretched, in use.

The conductive wires may be arranged to move from the non-linear configuration to a substantially linear configuration when the elastic substrate is stretched, in use.

For example, the stretchable electrical conductors may be arranged in a substantially sinusoidal shape within the elastic substrate. Advantageously, when the elastic substrate is stretched, the wires also stretch.

In yet another embodiment, the stretchable electrical conductors may comprise a woven cable or a helically wound cable.

The at least flexible circuit may be flexible within a range of 30% to 80%. For example, the at least partially flexible circuit may be at least 30% or 35% or 40% or 45% or 50% or 55% or 60% or 65% or 70% or 75% flexible. In other embodiments, the at least flexible circuit may be over 80% flexible.

The at least one stimulator may be adapted to be disposed at a predefined location relative to the part of the body.

The at least one stimulator may be adapted to provide electrical stimulus or mechanical stimulus. For example, the at least one stimulator may be a vibro-tactile stimulator or skin-stretch stimulator.

The device may further comprise at least one control unit operably connected with the at least one stimulator, the control unit configured to generate a control signal to control the at least one stimulator. The control unit may be disposed within the at least partially flexible circuit.

The control signal may be adapted to provide rhythmically modulated stimulation or constant stimulation. The control signal may be adapted to provide stimulation at a fixed intensity or at various intensities over the duration of the stimulation.

The at least partially flexible circuit may comprise a rechargeable power supply.

The rechargeable power supply may be configured for wireless charging.

The device may further comprise at least one sensor operably connected with the at least one control unit, the at least one sensor configured to receive monitoring data.

The at least on sensor may comprise a motion sensor.

In an embodiment, the at least one sensor may comprise a plurality of sensors, each sensor located at an anatomical marker, in use, and configured to obtain movement information of the body of the subject.

The monitoring data may comprise movement data of the body of the subject.

Movement data may comprise an indication of cadence, step-length, step-time variability, occurrence of freezing-of-gait (FOG), occurrence of limb tremor, dystonia or a pressure between an area of the foot of the subject and the floor.

The device may further comprise a processor configured to compare the obtained monitoring data with pre-determined movement information.

For example, the pre-defined information may be indicative of historical movement data of the subject. Alternatively, the pre-defined information may be indicative of another type of movement data.

Guiding movement of at least part of a body may include directing the part of the body to achieve a target movement or position.

Guiding movement may include directing the body to increase desirable movement and/or decrease undesirable movement.

Guiding movement of at least part of a body may include stimulation of the peripheral nervous system (PNS) to direct the part of the body to achieve a target movement or body position.

For example, the target movement may be stable gait.

In another example, the target movement may be reduced limb tremor.

In other examples, the target movement can include a movement that improves balance or strength.

In another aspect, there is provided a system for guiding movement of a part of a body of a subject, the system comprising:
at least one stimulation device for guiding movement of the part of the body of the subject;
a controller operably connected to the stimulation device for controlling the stimulation device;
at least one sensor for sensing monitoring data, and a processor for comparing the obtained monitoring data with pre-determined movement information to determine whether a desired movement has occurred.

The processor may be further configured to determine a further control signal upon determining that a desired movement has not occurred.

The system may further comprise a user device wirelessly connected to the stimulation device. The user device may be configured to display monitoring data and/or to transmit instructions to the controller to generate a control signal.

The user device may comprise:
a receiver for receiving monitoring data from the at least one sensor;
a processor for running a software application configured to generate a user interface including buttons for altering a control signal upon interaction with a user;
a display for displaying the monitoring data, and a transmitter for transmitting a control signal to the controller.

The at least one sensor may further comprise a pressure sensor configured to detect contact pressure between the at least one stimulator and the skin of the subject.

The at least one sensor may further comprise a pressure sensor configured to detect contact pressure between the flexible substrate and/or the skin of the subject.

In yet another aspect of the present disclosure, there is provided a method for guiding movement of at least part of a body, comprising:
providing the stimulation device;
generating a control signal to actuate a desired movement for a desired duration;
providing a stimulus to the body through the least one stimulator to guide the desired movement;
obtaining movement data of the body, and determining whether the desired movement has occurred.

Determining whether the desired movement has occurred may further comprise comparing monitoring data to the predetermined movement data.

The method may further comprise adjusting the control signal based on the obtained monitoring data.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

Other aspects of the invention are also disclosed.

### BRIEF DESCRIPTION OF THE DRAWINGS

Notwithstanding any other forms which may fall within the scope of the present invention, a preferred embodiments of the invention will now be described, by way of example only, with reference to the accompanying drawings in which:

FIG. 1A shows a stimulation device in accordance with an embodiment of the present invention;

FIG. 1B shows a stimulation device in accordance with another embodiment of the present invention;

FIG. 2A shows an example of a stretchable electric conductor in a reduced configuration in accordance with an embodiment of the present invention;

FIG. 2B shows an example of a stretchable electric conductor in an extended configuration in accordance with an embodiment of the present invention;

FIG. 3 shows examples of stretchable conductors in accordance with other embodiments of the present invention;

FIG. 4A shows a stimulation device in accordance with another embodiment of the present invention;

FIGS. 5A and 5C show a stimulation device in accordance with yet another embodiment of the present invention;

FIG. 6A shows a stimulation device for providing stimulation to a foot/ankle of a subject in accordance with yet another embodiment of the present invention;

FIG. 6B shows the location of stimulators in a stimulation device for providing stimulation to a foot/ankle of a subject in accordance with the embodiment of FIG. 6A of the present invention;

FIG. 7 shows a stimulation device for providing stimulation around a knee of a subject in accordance with yet another embodiment of the present invention;

FIG. 8 shows a stimulation device for providing stimulation around a hand and wrist of a subject in accordance with yet another embodiment of the present invention;

FIG. 9 shows a system for providing stimulation in accordance with an embodiment of the present invention;

FIG. 10 shows a method for providing stimulation in accordance with an embodiment of the present invention, and

FIG. 11 shows examples of stimulation devices in accordance with embodiments of the present invention.

### DESCRIPTION OF EMBODIMENTS

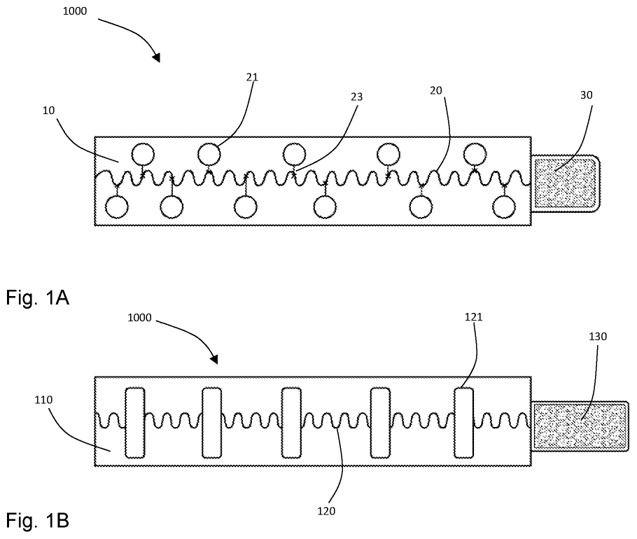

Stimulation devices 1000 in accordance with embodiments of the invention are shown in FIGS. 1A to 10. Devices, systems and methods for guiding movement of a body are described herein in relation to improving the gait of patients with Parkinson's disease. However, it is envisaged that the device 1000 can be used to improve the gait of patients experiencing impaired gait as a result of other diseases or neural impairment such as peripheral neuropathy, multiple sclerosis, cerebral palsy, stroke, spinal injury, or muscular distrophy. For example, the device can be used to address unstable gait, irregular stepping, freezing of gait, hypokinetic gait, gait festination, tremor, upper limb tremor, lower limb tremor, dyskinesia, contractures by guiding movement of the limbs of a subject.

As shown in FIG. 1A, the device 1000 includes a flexible substrate 10 configured to conform to an outer surface of a part of the body such as an ankle of a subject. The device 1000 also includes a stimulator circuit 20 at least partially embedded within the flexible substrate 100.

The stimulator circuit 20 comprises at least one stimulator 21 configured to extracorporeally provide stimulation to the part of the body around to guide movement.

The flexible substrate 10 and the stimulator circuit 20 are together configured to provide a uniform contact pressure on the skin when wrapped around a part of the body of the subject, in use.

The flexible substrate 10 can comprise an orthosis or other wearable garment, for example. The flexible substrate 10 can also be a stretchable bandage that can be wrapped around a part of the body in such a way as to provide an even contact pressure between the device and skin. Advantageously, this will help achieve good contact between each stimulator 21 and skin such that each stimulator 21 can effectively deliver stimulation to cue or otherwise cause a desired movement.

The flexible substrate 10 can be stretchable in one or more directions. For example, the flexible substrate 100 can be configured to stretch longitudinally and/or laterally.

In FIG. 1A, the flexible substrate 10 comprises an elastic material that is stretchable.

The flexible substrate 10 can comprise a stretchable bandage or other material that is moisture wicking, light weight, porous and breathable such as neoprene. For example, the flexible substrate can include stretchable Velcro.

In other embodiments the flexible substrate 10 can comprise a combination of materials.

The stimulator circuit 20 is at least partially flexible. The stimulator circuit 20 is configured to at least partially stretch in one or more directions. For example, the stimulator circuit 20 can stretch longitudinally and/or laterally relative to the flexible substrate 10 to provide sufficient extra length to ensure good contact between stimulators and the skin to effectively provide stimulation to the part of the body, in use.

The skilled person will understand that the stimulator circuit can be designed in a number of different ways to power the stimulators and to selectively deliver stimulation to the body of the subject.

Each of the plurality of stimulators is adapted to be disposed at a predefined location relative to the part of the body, in use. Each of the plurality of stimulators can be configured to deliver various types of stimuli. For example, the plurality of stimulators can provide an electrical stimulus or mechanical stimulus or a combination of the two. For example, the at least one stimulator may be a vibro-tactile stimulator or skin-stretch stimulator.

The device may further comprise at least one control unit operably connected with the one or more stimulators. The control unit is configured to generate a control signal to control the at least one stimulator in the stimulator circuit.

The flexible and stretchable circuit can include the control unit.

Advantageously, the flexible substrate 10 and the stimulator circuit 20 are together configured to provide effective stimulation yet be comfortably worn by the subject, in use.

As shown in the illustrated figures, the stimulator circuit 20 comprises rigid components 21 and stretchable components 23. The rigid 21 and stretchable components 23 can be arranged within the substrate 10 in such a way as to provide a desired amount of flexibility and stretch.

In embodiments, the flexible substrate 10 and stimulator circuit 20 can be flexible within a range of 30% to 80%. For example, the flexible substrate 10 and stimulator circuit 20 together can be 30% or 35% or 40% or 45% or 50% or 55% or 60% or 65% or 70% or 75% flexible. In other embodiments, the at least flexible stimulator circuit 20 can be over 80% flexible.

In embodiments, the flexible substrate 10 and stimulator circuit 20 can be stretchable or provide an increase in length by 30% to 80% in use. For example, the flexible substrate and stimulator circuit can be at least 30% or 35% or 40% or 45% or 50% or 55% or 60% or 65% or 70% or 75% stretchable.

The elasticity or degree of stretch of the flexible substrate, can be altered to maximize long-term comfort and minimize chaffing or other skin irritation that might be expected with daily ongoing use, while still maintaining a good connection between the surface of the body part and the stimulator, thus optimizing the effectiveness of the stimulation.

In the illustrated embodiments, the stretchable components 23 are stretchable electrical conductors that are moveable between a reduced configuration and an extended configuration.

For example, the stretchable electrical conductors 23 can be in a non-linear arrangement within the flexible substrate 10 and arranged to move from a non-linear configuration to a substantially linear configuration when the elastic substrate 10 is stretched, in use.

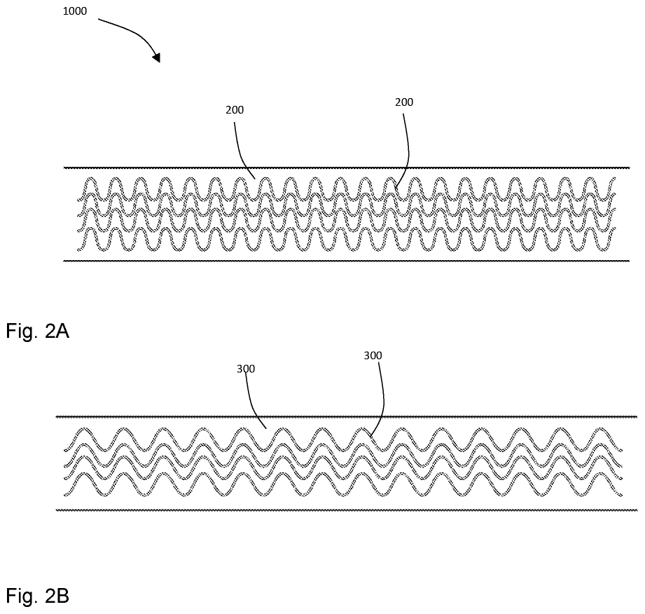

As shown in FIGS. 1A and 1B, the stretchable electrical conductors 23 comprise conductive wire arranged in a substantially sinusoidal shape within the substrate 10. FIG. 2A shows an example of conductors 23 in a reduced configuration. FIG. 2B shows the conductors 23 in an extended configuration when a user pulls the elastic material longitudinal direction.

The stretchable electrical conductors 23 comprise an elastic component. The elastic component of the stretchable electrical conductors 23 can be attached or otherwise fixed to the flexible and stretchable substrate 21 in a number of different ways. Alternately, the conductive wires can be sewn into, woven through or otherwise engaged with the substrate 10.

In embodiments 1A and 1B, the device 1000 is in the form of an elongated band which can be fastened around a body part of a subject. One end of the band can be attached to the other end via a fastener 30, such as Velcro, in use. It is envisaged that in other embodiments, the device 1000 can be fastened on the part of the body using other suitable fasteners.

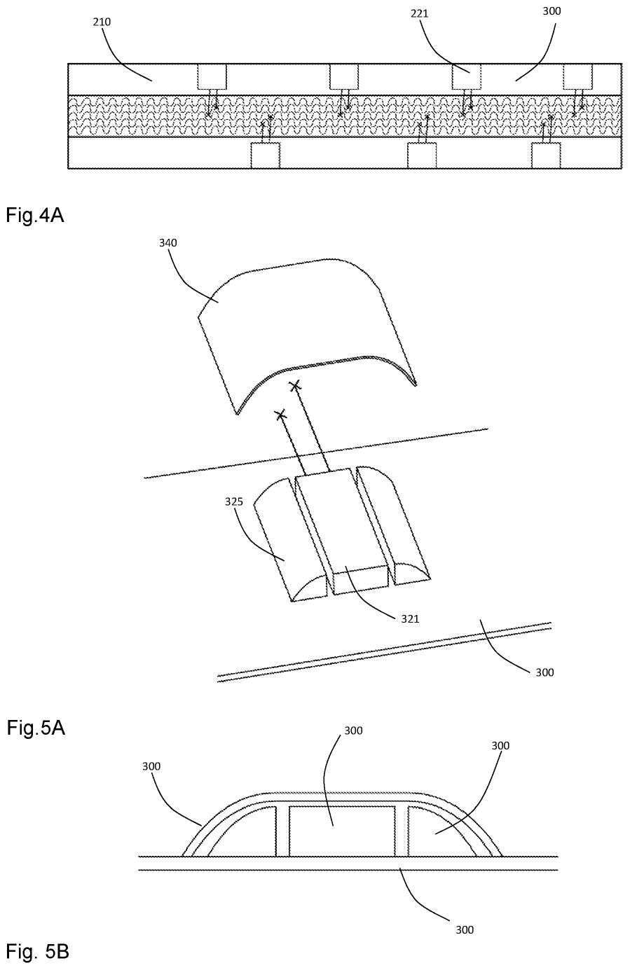

In the embodiments shown in 1A and 1B the conductors 20, 120 are located along a middle portion of the band and in FIG. 4A, the conductors 223 are located along a middle portion of an elongated portion of the device. In these embodiments, the conductors 223 extend along a length that is substantially the same as or greater than the length of the circuit.

In 1A and 4A, each of a plurality of stimulators 21, 221 and other rigid components 20, 220 are located perpendicularly to the conductors and are electrically connected to the stretchable conductors 223 via short conductive wires 24, 224 so that upon stretching of the band in the longitudinal direction, the stretchable conductors will stretch but the short conductive wires 224 will not be strained and therefore, not break. It is envisaged that the rigid and flexible circuit components can be arranged in other circuit configurations that provide flexibility and effective stimulation.

In another embodiment shown in FIG. 1B, the stimulators 211 or other rigid 210 electrical components are located at spaced intervals along the longitudinal direction of the band. In this embodiment, when the band is extended in the longitudinal direction the rigid components move away from each other along the longitudinal direction as the segments of the electrical conductors increase in length.

The stretchable electrical conductors 23 can extend across substantially the entire area covered by the circuit as shown in the embodiments of FIGS. 1A and 1B so that the elastic material and circuit can extend together when manually extended for example, by pulling in a direction by the user to wrap the device around the part of the body.

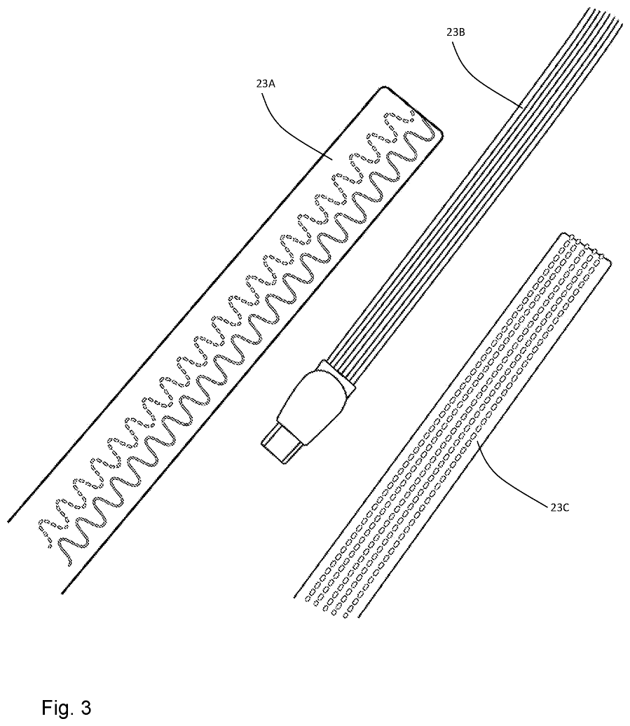

FIG. 3 shows examples of conductive conductors 23 which are configured to provide extra length when stretched. 23A shows conductive conductors in a sinusoidal shape which are woven through the elastic material. 23B shows conductive conductors arranged in parallel along a width of the substrate. 23C shows conductive wires sewn into the elastic material in a double helix.

23A, 23B, and 23C are configured so that undulations of the conductive wires extend perpendicularly to the direction of stretch in the reduced position of the conductors.

The conductive conductors can include relatively thin wires such as copper or silver thread. In another example, the conductive conductors can be helically wound to provide extra length in an axial direction of the helix.

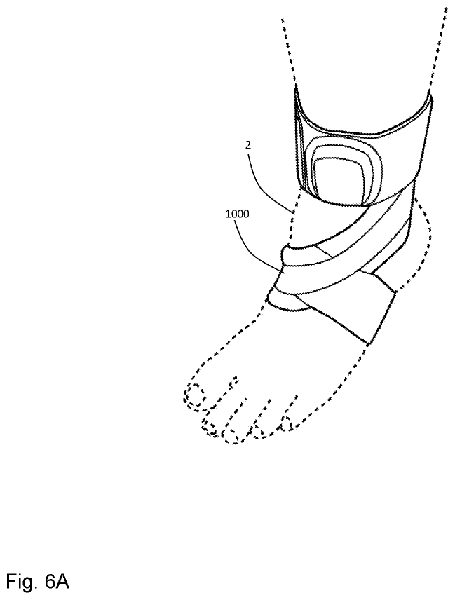

FIGS. 5A and 5B illustrate another embodiment of the device. In this embodiment, supports 325 are provided between rigid components 323. Advantageously, stresses effected by the band and rigid components on the patient's skin are distributed over a larger area and provide cushioning to the subject's limb. The supports 325 can comprise a relatively stiff sponge or other material which provides sufficient padding.

The padding is provided so as not to interfere with provision of stimulation, while the padding can be located all around each of the other rigid components.

In the embodiment shown in FIG. 5B, padding is provided on either side of a rigid component 321 and extends along a height of each of the stimulators.

Advantageously, this prevents stress concentration on parts of the subject's limb which may result from rigid components being pressed against the subject's skin over an extended period of time. Over time pressures sores can be caused in areas experiencing points of high applied pressure.

To construct this the rigid components 321 of the stimulator circuit 320 can be arranged on a sealed, liquid proof flexible substrate layer, and a settable material poured over the components embedding the circuit within the settable material. When the material is set, it can be covered with a cover layer.

An example of a settable material is epoxy resin, settable rubber, thermoplastic materials, or silicone rubber which can be cured, vulcanized or catalyzed.

As mentioned above, the flexible substrate can be provided by a wearable garment. In other embodiments, the simulator circuit be embedded unobtrusively into apparel worn by the subject. In some examples, the flexible substrate may be part of an item of apparel. Examples of apparel can include for example stimulating shoes, insoles, gloves, knee pads, gloves, hats, elbow pads, shorts, lumbar wraps, head bands, bras, or shirts, other tight-fitting clothing. The closeness of the apparel to the skin can help facilitate contact between the stimulators and the part of the body to which stimulation will be delivered.

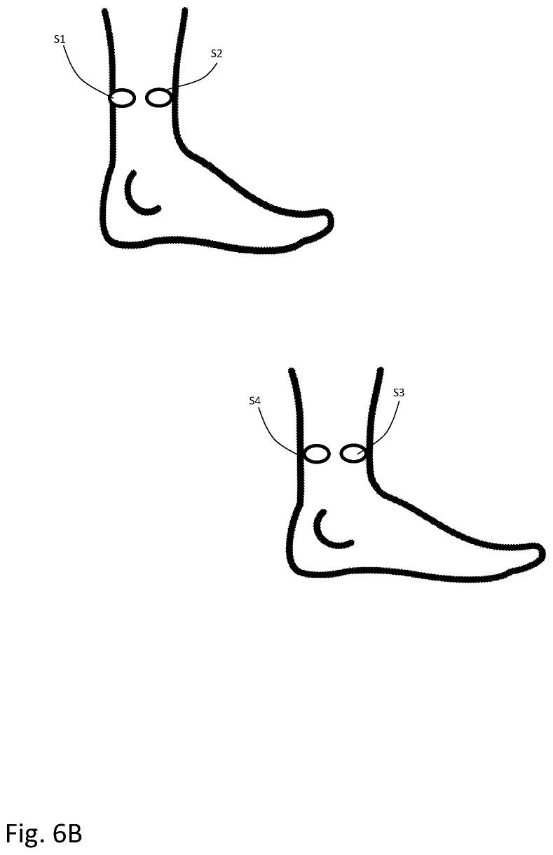

FIG. 6A shows an example of the device 1000 secured around the ankle 2 of the subject. In this embodiment, the device 1000 comprises a first band and a second band connected to each other so as to wrap around the foot and shin of the subject. For example, one or more stimulators can be a vibro-tactile stimulator located in a portion of the band located under the foot. In FIG. 6A, the control unit is located around in a band secured to the shin of the subject. Advantageously, the control unit does not protrude into the skin of the subject when worn.

In this example (as illustrated in FIG. 6B), the first band which extends around the shin of the subject also includes four vibrotactile stimulators, S1, S2, S3 and S4, each of which are positioned in pre-determined locations to selectively stimulate the body and therefore, cause a desired movement. The predetermined locations can include golgi tendon organs located in tendons between bone insertions and muscle bellies near points of articulation of joints.

S1 is positioned and otherwise configured to assist in causing plantar-flexion of the foot by stimulating the golgi tendon organs (proprioceptive sensors) found near the head of the fibularis and soleus muscles and gastrocnemius tendon of the subject.

S2 is positioned and otherwise configured to assist with dorsi-flexion of the foot by stimulating the golgi tendon organs (proprioceptive sensors) found near the head of the extensor digitorum longus muscle.

S3 is positioned and otherwise configured to assist with dorsi-flexion of the foot by potentially stimulating the golgi tendon organs (proprioceptive sensors) found near the head of the tibialis anterior muscle.

S4 is positioned and otherwise configured assist with plantar-flexion of the foot by potentially stimulating the golgi tendon organs (proprioceptive sensors) found near the head of the flexor digitorum longus, hallicis longus and soleus muscles and gastrocnemius and tibialis posterior tendons and tibial nerve.

As discussed further below, the device is configured selectively trigger one or more of the stimulators or example, in a specific sequence to trigger the desired movement.

Similarly, when it is desirable to cause a desired movement by stimulating the arch of the foot, stimulators can be placed near specific muscle heads, tendons or nerves which are known to play a role in causing the movement.

In embodiments in which the device includes electrical stimulators, the stimulators may be positioned near sensory receptors in the muscles responsible for joint articulation and stabilisation further from the point of articulation.

It is envisaged that in some examples, the device is configured such that the locations of each stimulator can be adjusted during use to selectively access and stimulate an anatomical area.

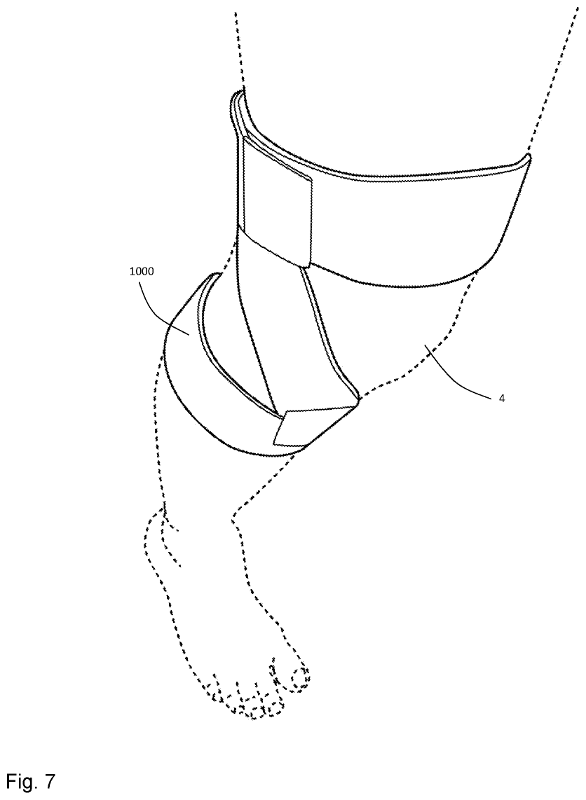

FIG. 7 shows an embodiment of the device 1000 configured to wrap around a knee 4 of a subject.

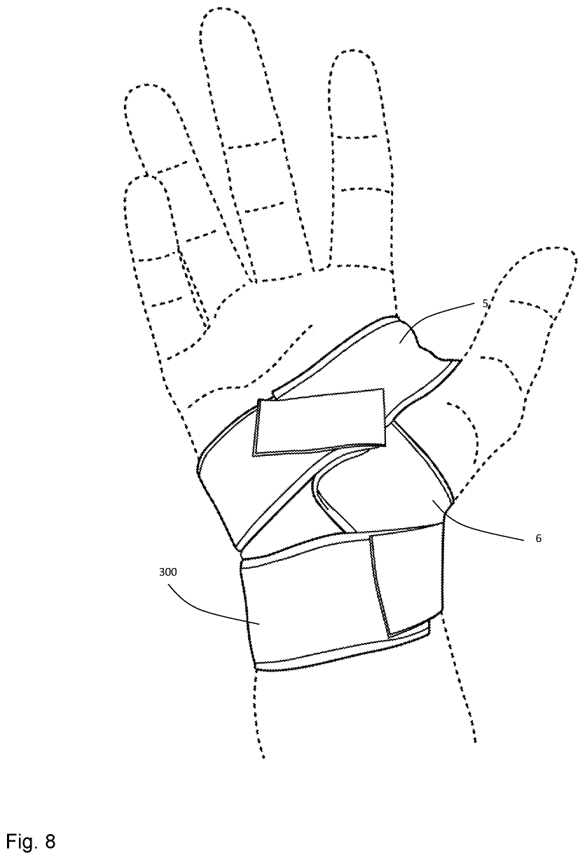

FIG. 8 shows another embodiment of the device 1000. This device 1000 is configured to wrap around a palm 5 and wrist 6 of a subject.

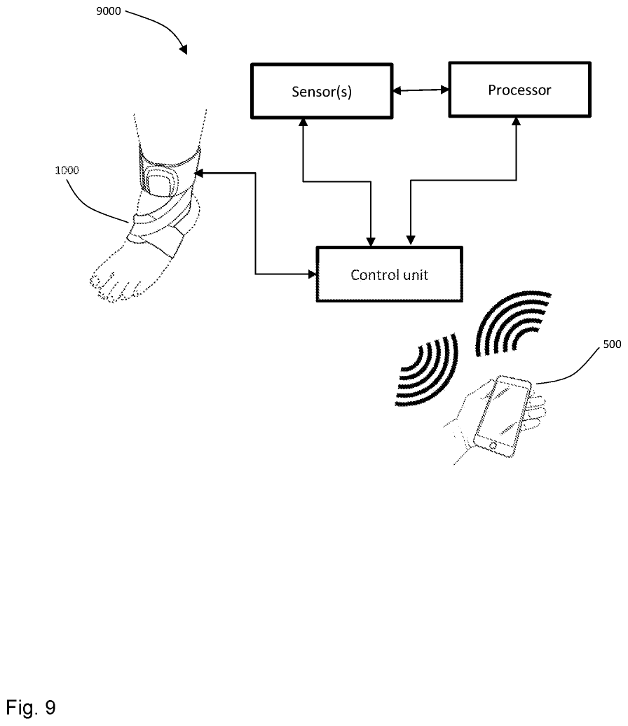

FIG. 9 illustrates a system 9000 for guiding movement of a part of a body of a subject.

The system 900 includes a stimulation device 1000 for guiding movement of the part of the body of the subject as described above. The system 9000 also includes a control unit/controller 2000 operably connected to the stimulation circuit of the device 1000 for controlling delivery of stimulus to the body part. The system includes a processor 3000 operably connected to the control unit 2000.

The controller 2000 can selectively trigger one or more stimulators to deliver a stimulus upon receiving an indication of the stimulus to be delivered from the processor 3000. In this embodiment the control unit 2000 is located externally to the device. However, in an embodiment the control unit can be included within the stimulation circuit of the device 1000.

The system 9000 also includes at least one sensor 4000 for sensing monitoring data. The system 9000 can include a plurality of sensors e.g. motion sensors that are placed on a subject's body to track the movement of a subject for example, in a gait lab.

In an embodiment, the at least one sensor 4000 may comprise a plurality of sensors, each sensor located at an anatomical marker, in use, and configured to obtain movement information of the body of the subject.

The monitoring data is obtained from the sensors and transmitted to a processor or stored in a database.

Movement data can include an indication of cadence, step-length, step-time variability, occurrence of freezing-of-gait (FOG), occurrence of limb tremor, dystonia or a pressure between an area of the foot of the subject and the floor.

The processor 3000 is configured to compare the obtained monitoring data with pre-determined movement information to determine whether the desired movement has occurred.

The processor 3000 is configured to compare the obtained monitoring data with pre-determined movement information upon receiving the monitoring data from the sensors. The data can be transmitted from the sensors wirelessly via Bluetooth, for example, or other suitable wireless means.

Monitored data can be received and stored in a database for example, hosted by a cloud or on a memory in the computer.

For example, the pre-defined information is indicative of historical movement data of the subject. Alternatively, the pre-defined information can be indicative of another type of movement data. The pre-defined information can be stored in and retrieved from a database.

Guiding movement of at least part of a body can include directing the part of the body to achieve a target movement or position.

Guiding movement can include directing the body to increase desirable movement and/or decrease undesirable movement.

Guiding movement of at least part of a body can include stimulation of the peripheral nervous system (PNS) to direct the part of the body to achieve a target movement or body position.

For example, the target movement can be stable gait. In another example, the target movement can be reduced limb tremor. Other examples can include running, swimming, cycling, dancing, sit to stand transitions, turning, stair ascent/decent, jogging, juggling, rowing, arms swings, leg swings, sit ups, crunches and figure-eight walking.

Other examples of target movement can include a movement that improves balance or strength.

For example, this may include tandem stance, isolated stance, standing on tip toes, chair pose, squats, warrior pose, leg curls, bicep curls, pull ups, push ups, and/or jumps.

If the target movement is not achieved, the processor can determine a further control signal to apply to the part of the body to achieve the desired movement.

As shown in FIG. 9, the system 9000 comprises a user device 5000 wirelessly connected to the stimulation device. The user device 5000 can be configured to display monitoring data and/or to transmit instructions to the controller to generate a control signal.

The user device 5000 can include a receiver for receiving monitoring data from the at least one sensor; the processor for running a software application configured to generate a user interface including buttons for altering a control signal upon interaction with a user; a display for displaying the monitoring data and a transmitter for transmitting a control signal to the controller.

For example, the user device 5000 can be a mobile phone or a desktop or laptop computer or other suitable device such as a smart watch.

The system 9000 can include at least one pressure sensor located at the interface between the skin and the device configured to detect contact pressure at the interface. The pressures sensor can be part of the stimulation circuit.

The stimulation circuit also includes a power supply. The power supply can be a battery and located within the substrate. The power supply can be a rechargeable battery located within the flexible substrate of the device. The power supply can be configured for induction charging.

In another embodiment the device includes a charging port for receiving a cable that is connectable to a mains power supply to charge the power supply of the stimulator circuit.

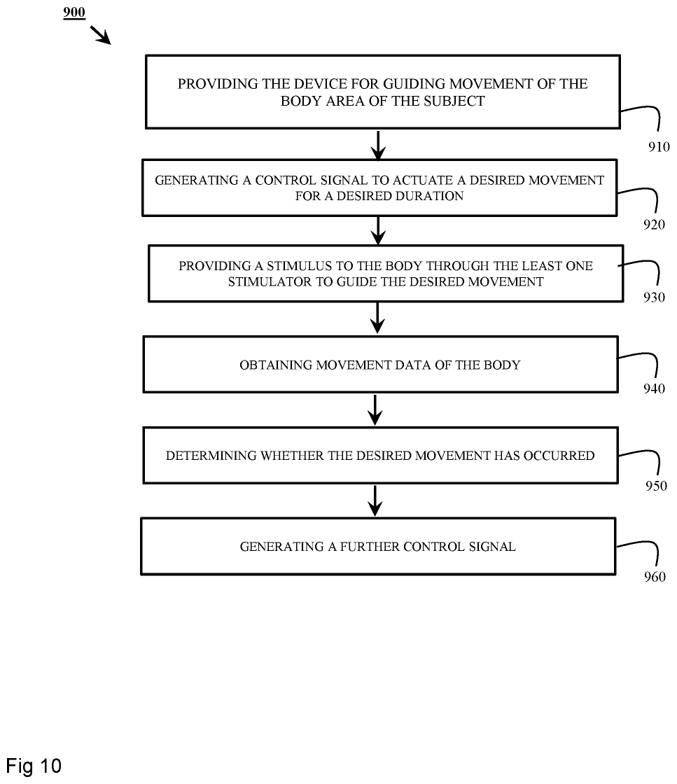

FIG. 10 illustrates a method 900 for guiding movement of at least part of a body using the stimulation system 9000 described above. At step 910 the device for guiding movement of the body of the subject is secured to the subject, operably connected to other system components and energized.

At step 920 a control signal is generated to actuate a desired movement for a desired duration.

At step 930 a stimulus is provided to the body through the least one stimulator to guide the desired movement. The subject is cued or otherwise caused to move.

At step 940 movement data of the body is obtained from sensors. During this step the sensors detect the cued or caused movement of the subject. The data is transmitted to a processor which assimilates the movement data to determine the gait of the subject. This can be shown as a 3D model of the subject's limbs moving over a period of time.

At step 950 whether the desired movement has occurred is determined by the user.

Determining whether the desired movement has occurred further comprises comparing the obtained monitoring data to the predetermined movement. For example, the recorded gait of the subject is compared to a desired gait or other movement of the subject.

At step 960 the method further comprises adjusting the control signal based on the obtained monitoring data to deliver a different stimulation directed to achieving the desired movement.

The device is adapted to operate in multiple control modes. For example, full manual control of the device enables the patient to turn stimulation on and off, and adjust stimulation frequency, duty cycle, and strength.

In open control mode, for example, once an intention to move in a certain way is determined from information from sensed movement, stimulation is provided in a fixed manner until the movement ceases.

In adaptive control, the control signal can be modified after monitoring of initial movements of the patient information is received. For example, if it is determined that a change in walking cadence is required, the controller uses feedback from the sensors to update the stimulation settings while the patient is moving. It is envisaged that the present invention can be used in conjunction with other cues such as auditory, visible or other sensory stimulus.

In an example, the stimulation device can be used in gait retraining. This involves interval walking at several different cadences with rest periods in between. Cadences may for example be slow (for example 20% slower than preferred cadence), at the preferred cadence, and fast (for example 20% faster than preferred cadence). Stimulation is provided to achieve the target cadence. Visual cues such as floor markings can be used in addition to the stimulation to achieve the goals of treatment. For example, visual cues can include a series of parallel lines to step over to help with gait initiation, or an arrangement of wagon wheel spokes can help with achieving a turn of consistent radius and consistent step lengths.

More challenging scenarios can include targets that must be selectively stepped on or avoided, obstacles to step over, gates to pass through and markers to turn around.

These visual cues can also be provided by augmented reality for example. Cues can also include external forces e.g. perturbations provided by a treadmill or a perturbation walkway.

Step training can involve more complex stepping patterns in different directions and can use visual cues such as a step mat and/or augmented reality, or barriers that need to be stepped over. Simulation can be provided at the target slow (for example 20% slower), preferred, and fast (for example 20% faster) cadences during training.

Dynamic training also includes repetitive training movements such as leg or arm curls with or without weights or resistance. Sit to stands for example can also be done to the desired training stimulation cadence.

Static training can involves causing the subject to perform balance poses or the static holding of weights. This can include standing on tip toes, star pose, a sitting pose or on a single leg, etc. Stimulation is timed with the duration the patient is told to hold the pose. The stimulation can be applied to multiple body parts and multiple locations on each body part simultaneously.

Auditory stimulation can also be provided in addition to vibration stimulus. For example, the stimulation can be in the form of a metronome beat synchronised with the duty cycle of the stimulation. The auditory stimulus can be provided by external speakers, ear buds, or bone conduction speakers.

For example, auditory stimulation can be in the form of foot fall sounds, which may activate mirror neurons during the participants walk, or set to match the participants preferred stimulation frequency e.g. at 220 Hz=middle C (or harmonics of the participants preferred stimulation frequency between 100-300 Hz) or a combination of both foot fall sounds and harmonics of the participants preferred stimulation frequency.

Electrical stimulation can be provided in addition to vibration stimulation. For example, electrical stimulation with a burst frequency of 100-300 Hz can be applied. In an example, the electrical stimulation can be synchronised to vibration.

Electrical stimulation can be provided above the sensory threshold (e.g. it can be felt) but below the functional threshold (e.g. it does not cause involuntary limb movement). Electrical stimulation can cause high frequency micro contractions of muscle fibres.

The electrical stimulation can be provided at motor neurons that directly activate muscle fibres through a monosynaptic loop, sensory neurons that regulate the h-reflex through a spinal loop, and sensory receptors which map to higher level structures in the spinal cord and central nervous system (CNS).

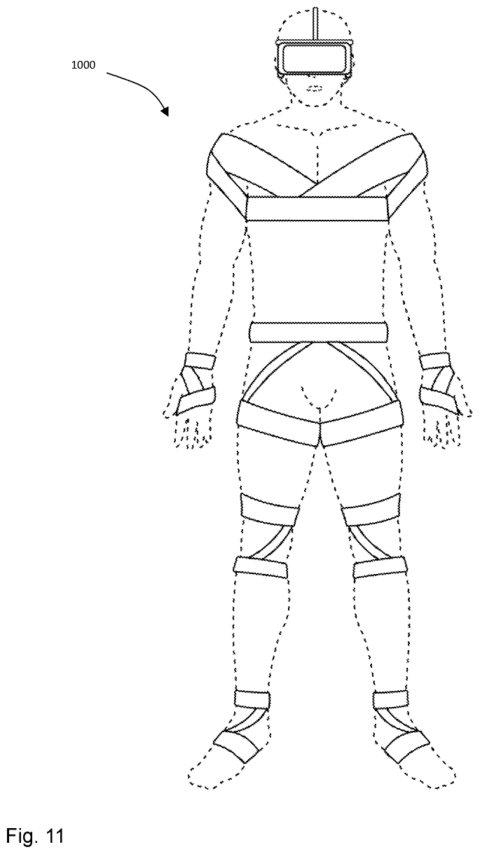

FIG. 11 illustrates various embodiments of the present invention configured to provide stimulation at various joints of the body such as the shoulders and the hips, knees, ankles and hands of a subject.

It is envisaged that the various devices can be used to act in concert by for example, being operated in a tailored sequence, to trigger or cause full body movement.

For example, rhythmic stimulation can be applied to multiple body parts simultaneously or in a coordinated manner to for example, to cause stabilized arm and leg movements.

For example, a control signal sequence can be wirelessly communicated to the various devices secured to the body to cause synchronised stimulation of the body. The patient's body can receive stimulation from multiple points on a body part and/or to multiple body parts at the same time.

For example, to control multiple devices to deliver synchronized stimulation, each stimulation device may be configured to deliver stimulation at the same stimulation frequency (or harmonic of the stimulation frequency). The stimulation frequency can be selected to suit the individual's neuromuscular system, and the stimulation duty cycles can be precisely coordinated to cause the target movement.

### EMBODIMENTS

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description of Specific Embodiments are hereby expressly incorporated into this Detailed Description of Specific Embodiments, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

### Comprising and Including

In the claims which follow and in the preceding description of the invention, except where the context requires otherwise due to express language or necessary implication, the word “comprise” or variations such as “comprises” or “comprising” are used in an inclusive sense, i.e. to specify the presence of the stated features but not to preclude the presence or addition of further features in various embodiments of the invention.

Any one of the terms: including or which includes or that includes as used herein is also an open term that also means including at least the elements/features that follow the term, but not excluding others. Thus, including is synonymous with and means comprising.

### Scope of Invention

Thus, while there has been described what are believed to be the preferred embodiments of the invention, those skilled in the art will recognize that other and further modifications may be made thereto without departing from the spirit of the invention, and it is intended to claim all such changes and modifications as fall within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to the medical and health industries.

1-31. (canceled)

32. A stimulation device for guiding movement of one or more parts of a body of a subject, the system comprising:
at least two stimulation devices, each stimulation device positioned on the one or more body parts, in use, each device comprising:
a flexible substrate configured to at least partially conform to an outer surface of the part of the body, in use, and
a stretchable stimulator circuit at least partially embedded within the flexible substrate, the stimulator circuit comprising at least one stimulator configured to provide a mechanical vibration stimulus to a part of a body to guide movement of that body part; and
a controller operably connected to each stimulation device for controlling each stimulation device,
wherein the system is adapted to deliver the mechanical vibration stimulus in a coordinated manner to the one or more body parts to guide movement of the one of more body parts.

33. The stimulation system of claim 32, further comprising at least one other stimulation device, wherein the at least one stimulator is configured to provide an electrical vibration stimulus to a part of a body to guide movement of that body part.

34. The stimulation system of claim 32, wherein the part of the body may be one or more of the following: nerves, muscles, tendons or golgi tendon organs.

35. The stimulation system of claim 32, wherein the flexible substrate is stretchable in one or more directions.

36. The stimulation system of claim 32, wherein the stimulator circuit is configured to at least partially stretch in one or more directions

37. The stimulation system of claim 32, wherein the stimulator circuit comprises stretchable electrical conductors extending between rigid electrical components.

38. The stimulation device of system 32, wherein the stretchable electrical conductors are moveable between a reduced configuration and an extended configuration.

39. The stimulation system of claim 32, wherein the stretchable electrical conductors comprise conductive wires in a reduced or non-linear arrangement so as to extend in a direction in which the elastic substrate is stretched, in use.

40. The stimulation system of claim 32, wherein the conductive wires are arranged to move from the non-linear configuration to a substantially linear configuration when the elastic substrate is stretched, in use.

41. The stimulation system of claim 32, wherein the stretchable electrical conductors are arranged in a substantially sinusoidal shape within the elastic substrate.

42. The stimulation system of claim 32, wherein the flexible substrate comprises one of the following: an orthosis or a stretchable bandage or a wearable garment or a band or a strap.

43. The stimulation system of claim 32, wherein the at least one stimulator is adapted to be disposed at a predefined location relative to the part of the body, in use.

44. The stimulation system of claim 32, wherein the at least one stimulator is adapted to provide electrical stimulus.

45. The stimulation system of claim 32, wherein the at least one stimulator is adapted to provide mechanical stimulus.

46. The stimulation system of claim 32, further comprising padding to cushion the part of the body.

47. The stimulation system of claim 32, wherein the control unit is part of the at least partially flexible circuit and located within the flexible substrate.

48. The stimulation system of claim 32, wherein the at least partially flexible circuit comprises a rechargeable power supply.

49. The stimulation system of claim 48, wherein the rechargeable power supply is configured for wireless charging.

50. The stimulation system of claim 32 further comprising at least one sensor operably connected with the at least one control unit, the at least one sensor configured to receive monitoring data.

51. The stimulation system of claim 50 wherein the at least one sensor is a pressure sensor configured to detect a contact pressure at the interface between the flexible substrate and skin of the subject, in use.

52. The system of claim 51 wherein the monitoring data comprises movement data of the body of the subject.

53. The system of claim 52 wherein the movement data may comprise an indication of cadence, step-length, step-time variability, occurrence of freezing-of-gait (FOG), occurrence of limb tremor, dystonia or an interface pressure between an area of the foot of the subject and the floor.

54. The system of claim 52 wherein the device further comprises a processor configured to compare the obtained monitoring data with pre-determined movement information.

55. The system of claim 54 wherein the pre-determined movement information is indicative of historical movement data of the subject.

56. The system of claim 54 wherein the processor is further configured to determine a further control signal upon determining that a desired movement has not occurred.

57. The system of claim 32 further comprising a user device wirelessly connected to the stimulation device, configured to display monitoring data and/or to transmit instructions to the controller to generate a control signal.

58. The system of claim 53, wherein the system comprises two or more stimulation devices that are adapted to deliver stimulus in a synchronized manner to multiple body parts to guide movement of multiple body parts.

59. A method for guiding movement of at least part of a body, comprising:
providing the stimulation system of claim 32;
generating a control signal to actuate a desired movement for a desired duration;
providing a stimulus to the body through the least one stimulator to guide the desired movement;
obtaining movement data of the body; and
determining whether the desired movement has occurred.

60. The method of claim 59 wherein determining whether the desired movement has occurred may further comprise comparing monitoring data to the predetermined movement data.

61. The method of claim 60 further comprising adjusting the control signal based on the obtained movement data.
